(19) United States
(12) Patent Application Publication
(10) Pub. No.: US 2025/0025177 A1
(43) Pub. Date: Jan. 23, 2025

(54) LEFT ATRIAL APPENDAGE EXCLUSION METHODS AND DEVICES

(71) Applicant: Maurice Buchbinder, San Diego, CA (US)

(72) Inventor: Maurice Buchbinder, San Diego, CA (US)

(21) Appl. No.: 18/775,566

(22) Filed: Jul. 17, 2024

Related U.S. Application Data
(60) Provisional application No. 63/514,114, filed on Jul. 17, 2023.

(51) Int. Cl. A61B 17/12; A61B 17/00

(52) U.S. Cl. CPC A61B 17/12031; A61B 17/00234; A61B 17/12122; A61B 17/12172; A61B 2017/00004; A61B 2017/00243; A61B 2017/00292; A61B 2017/1205

(57) ABSTRACT

Left atrial appendage exclusion devices and methods of excluding a left atrial appendage. The left atrial exclusion devices can be carried by a flexible elongate member, have an invaginating member, and have a plurality of flexible elongate members forming a distally open configuration. The invaginating members invaginate the left atrial appendage, which is then sutured closed or covered and left in the left atrium of the LAA ostium.

### BACKGROUND OF THE INVENTION

### Field of the Invention

The invention related to left atrial appendage closure devices and methods of employing them.

### Background Art

The left atrial appendage (“LAA”) is a small sac in the muscle wall of the left atrium. It is unclear what function, if any, the LAA performs. In normal hearts, the heart contracts with each heartbeat, and the blood in the left atrium and LAA is squeezed out of the left atrium into the left ventricle.

Atrial fibrillation (AF) is the irregular, chaotic beating of the upper chambers of the heart. Electrical impulses that control the heartbeat do not travel in an orderly fashion through the heart. Instead, many impulses begin at the same time and spread through the atria. The fast and chaotic impulses do not give the atria time to contract and/or effectively squeeze blood into the ventricles. As a result, the blood is not squeezed from the LAA in regular heartbeats. Because the LAA is a little pouch, blood collects there and can form clots in the LAA and atria. When blood clots are pumped out of the LAA, and then out of the heart, they can cause a stroke.

It is estimated that AF patients have five times the stroke risk of patients without AF. Most AF patients, regardless of the severity of their symptoms or frequency of episodes, require treatment to reduce the risk of stroke. In non-valvular AF, over 90% of stroke-causing clots that come from the left atrium are formed in the LAA.

The most common treatment for stroke risk reduction in patients with AF is blood-thinning therapy with oral anti-coagulants. Oral anti-coagulants effectively reduce the risk of cardioembolic stroke and are the most commonly used treatments in at-risk patients with non-valvular atrial fibrillation. Many patients have concerns about, or dislike, taking oral anti-coagulants. Some of the reasons for this are: Frequent blood draws are needed to measure the patient's international normal ratio (INR), or clotting time. The tests are needed to make sure the patient takes the right amount of medication; while taking warfarin, you need to limit your intake of certain foods that contain vitamin K; the risk of bleeding is higher while taking oral anti-coagulants; and some patients do not tolerate medical therapy.

Thus, one alternative treatment is to perform a LAA closure. While it is common to perform a LAA closure in AF patients, a LAA closure can also benefit patients who need heart surgery, or other risk factors for a stroke.

There has thus been a desire to attempt to filter, occlude and/or isolate the LAA to prevent clots from forming therein, which can be subsequently released from the LAA and cause a stroke. It is also desirable to occlude the LAA to isolate blood clots that may already be forming in the left atrial appendage.

There are devices on the market that are adapted to filter and/or occlude the LAA to reduce the likelihood of stroke. For example, the Watchman™ device (FDA approved in 2015) is implanted in the left atrial appendage, and initially acts as a filter between the LAA and the atria to prevent clots from being released from the LAA. Over time, cells grow over the device, effectively sealing off the LAA from the atrium. U.S. Publication 2016/0058539, including all of its methods of delivering an occlusion device to the LAA, are incorporated by reference herein.

The anatomy of the LAA is not consistent from one patient to the next. The LAA can have substantially different sizes from one patient to the next. The opening can also be highly irregular. As a result, current approaches require many differently sized and shaped implants available for implantation depending on the anatomy of a particular patient, which is generally assessed prior to implantation using imaging techniques, such as ultrasound imaging techniques (e.g., TEE) and computerized tomography (CT). There is a need for modified and improved method to close off the LAA and prevent strokes.

### BRIEF SUMMARY OF THE INVENTION

The present invention solves these needs by providing a system for deploying a left atrial appendage closure device that includes a delivery catheter that has a lumen, an invagination catheter, the invagination catheter comprising: arms, the arms having barbs for piercing the left atrial appendage; the invagination catheter inside the lumen of the delivery catheter, and movable relative to the delivery catheter; a looping device comprising a suture loop; a suture knot; and a cutting device.

In another embodiment the system is designed for deploying a left atrial appendage exclusion device, the system including a delivery catheter, the delivery catheter comprising a lumen; an invagination catheter, the invagination catheter comprising arms, the arms having barbs for piercing the left atrial appendage; the invagination catheter inside the lumen of the delivery catheter, and movable relative to the delivery catheter; a disk assembly comprising a fabric material; a frame, attached to the fabric material; a disk catheter, the disk catheter removably attached to the frame.

In one embodiment the system includes a hub configured to connect the arms to the delivery catheter. In another embodiment the system includes a connector configured to attach the disk assembly to the hub. The connector may be a tube and may have a lip that frictionally mates with a cutout on the disk assembly.

In some embodiments there may be a threaded portion on the disk assembly and a threaded portion on the disk catheter, the threaded portion on the disk catheter removably connected to the threaded portion on the disk assembly.

In some embodiments, one or more of the barbs, arms, fabric material, or frame may be a bioabsorbable material. The bioabsorbable material may be a PLLA, or PLGA material.

In some embodiment the invagination catheter is a suture. In others, the suture may be braided or comprised of a bioabsorbable material.

In some embodiments the frame is between 20 and 25 mm in diameter.

In other embodiments the invention is a method of invaginating a left atrial appendage, the method comprising providing a delivery catheter, the delivery catheter comprising a lumen; an invagination catheter, the invagination catheter comprising arms, the arms having barbs for piercing the left atrial appendage; the invagination catheter inside the lumen of the delivery catheter, and movable relative to the delivery catheter; a disk assembly comprising a fabric material; a frame, attached to the fabric material; a disk catheter, the disk catheter removably attached to the frame; inserting the invagination catheter into the LAA; piercing the LAA with the barbs invaginating the LAA by partially removing the invagination catheter proximally covering the ostium of the LAA with the disk assembly.

### BRIEF DESCRIPTION OF THE DRAWINGS

FIG. 1 is a partial perspective view of a left atrial appendage exclusion device;

FIG. 2 is a partial perspective view of a left atrial appendage exclusion device;

FIG. 2a is a cross sectional view of an embodiment of a left atrial appendage exclusion device;

FIG. 2b is a cross sectional view of an embodiment of a left atrial appendage exclusion device;

FIG. 2c is a cross sectional view of an embodiment of a left atrial appendage exclusion device;

FIG. 3 is a partial perspective view of a left atrial appendage exclusion device;

FIG. 3a is a partial perspective view of a left atrial appendage exclusion device;

FIG. 4 is a partial perspective view of the closure portion of a left atrial appendage exclusion device of the present invention;

FIG. 5 is a partial perspective view of the closure portion of a left atrial appendage exclusion device of the present invention;

FIG. 6 is a partial perspective view of the closure portion and the covering portion of a left atrial appendage exclusion device of the present invention;

FIG. 7a is a cross sectional view of a pair of grabbers and a connector of the present invention;

FIG. 7b is a cross sectional view of a pair of grabbers and a connector of the present invention;

FIG. 7c is a cross sectional view of a pair of grabbers and a connector of the present invention;

FIG. 8 is a partially exploded partial perspective view of a grabber assembly of the present invention;

FIG. 9 is a partial perspective view of a disk assembly and a grabber assembly of the present invention;

FIG. 10 is a cross-sectional view of a disk assembly of the present invention;

FIG. 11a is a partial perspective view of a disk assembly and a grabber assembly of the present invention before being linked;

FIG. 11b is a partial perspective view of the disk assembly and a grabber assembly of FIG. 11a, of the present invention after being linked.

### DETAILED DESCRIPTION

The disclosure generally relates to methods and devices for closing a left atrial appendage (“LAA”).

Some aspects of the disclosure relate to catheters, sheaths, and associated devices adapted, sized and configured for LAA exclusion or LAA obliteration. In one aspect the invention invaginates or partially invaginates the LAA. In other aspects the invention seals off the ostium of the LAA. Some aspects of the different embodiments herein, however, may be suitable for incorporation into different embodiments, including devices and methods.

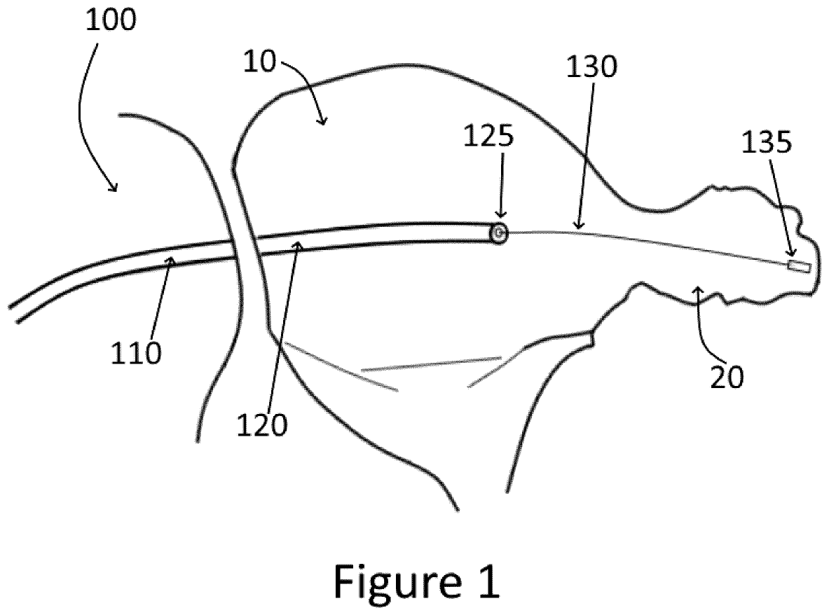

FIGS. 1-5 illustrate a left atrial appendage exclusion device 100 for occluding a left atrial appendage. Exclusion device 100 includes external portion (not shown) that may include a handle, actuators, ports, and stabilizing mechanisms. The external portion is coupled to elongate member 110 that comprises shaft 120, and distal member 130 that is carried inside and exits from a distal region 125 of shaft 120. Distal shaft 130 is carried to the left atrium 10 by shaft 120, and typically in a lumen (not shown) of shaft 120. Distal shaft 130 may have an atraumatic tip 135 to prevent unwanted perforations, both in the LAA 20 and the left atrium 10.

Shaft 120 may be a sheath, for example, and may range in diameter from 8-20 french, e.g., 14 french. The sheath may have one or more preformed bends, e.g., a single preformed bend or two preformed bends that will orient its distal portions toward the fossa ovalis when delivered from a femoral vein access. Alternatively, the sheath may have pull wires or other steering mechanisms to help orient it in the desired direction and location.

In FIG. 1, distal shaft 130 is shown extending out of the lumen in shaft 120. However, during initial delivery to the LAA, the distal shaft 130 may be mostly or entirely within a lumen of shaft 120. Shaft 120 is delivered to the left atrium via a typical transseptal access. In one common approach a long sheath with a dilator is introduced through the femoral vein and advanced into the superior vena cava over a guide wire. The fossa ovalis is crossed via a BRK needle. The needle is removed and a guidewire, e.g., a 0.035″ diameter guidewire, is left in the left atrium. The delivery system consists of a dilator and a curved or steerable sheath, which can include shaft 120, or shaft 120 can be delivered to the left atrium inside the steerable sheath. The sheath may be precurved as shown in FIG. 1, may be steerable via pull wires, magnetic guidance, or have a steering element that may be inserted to direct the delivery system to the fossa ovalis. The dilator passes through the fossa ovalis, is followed by the sheath, and is then typically removed. In one embodiment though, the dilator can also serve as an atraumatic tip, or can serve as a hub as described below. The guidewire is advanced into the LAA. At this point the delivery system is in place.

Once the system is in the left atrium the distal region 125 of shaft 120 is navigated to in or near the LAA, unless it is already in place. At this point the distal shaft 130 is advanced out of the distal region 125, either by an actuator that, upon actuation advances the distal shaft 130 out of the distal region 125, or by simply pushing a proximal end (e.g., outside the body) of distal shaft 130 further into the proximal end/handle of shaft 120.

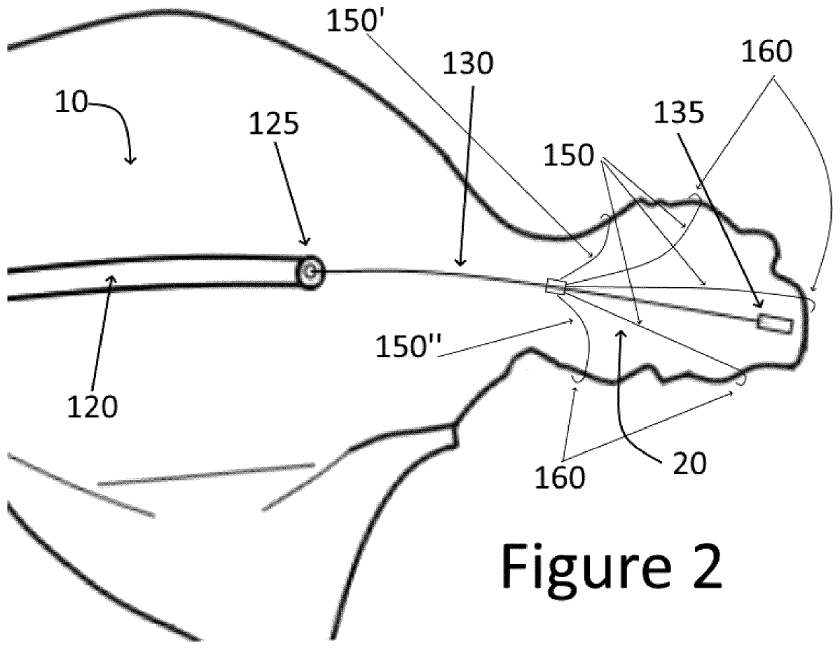

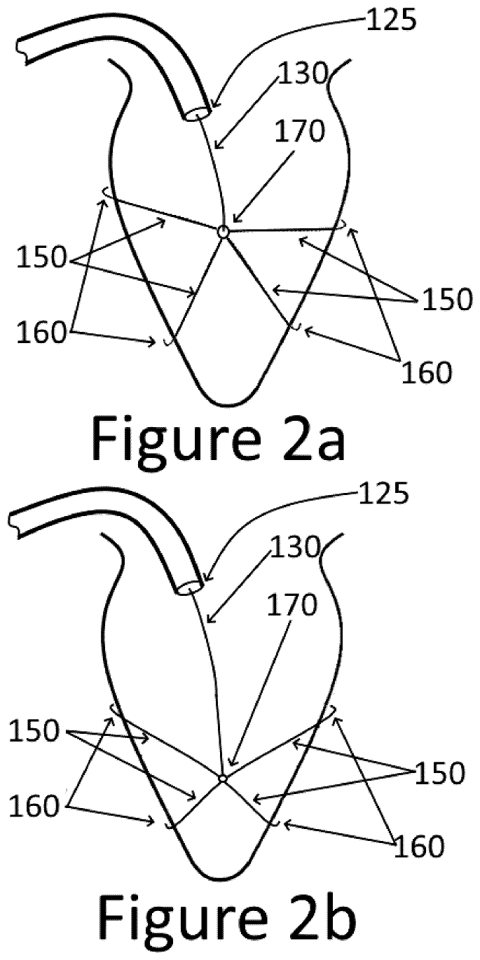

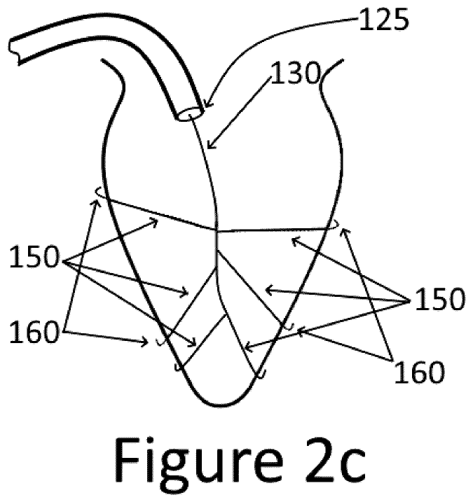

As shown in FIG. 2, in one embodiment as the distal shaft 130 is advanced a series of arms 150 are freed or actuated and extend laterally away from the distal member 130's shaft. Arms 150 may have barbs 160 on their distal end. Barbs 160 may be replaced by other mechanisms to grab tissue, such as a screw, an anchor, a hook, a pigtail, or adhesives in other embodiments. The arms 150 may be all the same length. As shown in FIG. 2a, the hub 170 can be advanced into the LAA with the arms extending outward laterally into different portions of the LAA, some to the more distal portion of the LAA, some to more proximal portions. In the embodiment shown in FIG. 2a the arms 150 are roughly equivalent in length, but the more proximal arms extend farther out from the coaxial center of the catheter shafts, 120, 130. Thus, the more distal arms extend largely forward to the most distal portion of the LAA, while other arms extend more laterally, or even proximally to a more medial or proximal portions of the LAA. Once a sufficient number of the barbs 160 are in the LAA tissue, the system is withdrawn toward the left atrium to pull all or part of the LAA into the left atrium, inverting or invaginating it. In this embodiment, when the system is withdrawn the more distally extending arms will pull the more distal portion of the LAA first, and as the system is withdrawn subsequent portions of the LAA, moving from the proximal side to the distal side, are inverted and pulled into the left atrium.

In the embodiment shown in FIG. 2b, the hub 170 is advanced farther into the LAA, and the arms 160 are of different lengths. In this embodiment, the shorter length of the more distally extending arms 160 contribute to their pulling the distal portion of the LAA first, before the proximal portions of the LAA wall are pulled.

Thus, in an embodiment the arms 150 have different lengths. Arm 150 may be 35 mm in length, while arm 150′ is 25 mm in length, and arm 150″ is 10 mm in length. This will allow different arms to advance to different depths within the LAA. So, for example, there may be one each arm 150 of 45 mm, 40 mm, 35 mm, 30 mm, 25 mm, 20 mm, 15 mm, 10 mm, and 5 mm, for example. The exact length of each arm is subject to the operator's requirements, the location of the hub 170, the depth of the hub or other attachment point, and can be varied. The length needed will also depend on the size of the LAA and how far into the LAA the distal region 125 is to be advanced. Thus, different portions of the LAA may be folded into the LA first. If the most distal portion is to be pulled or folded first, the arms that engage his portion may be shorter, or in another embodiment may be longer, but extend straight forward from a hub positioned outside the LAA. If the proximal portion of the LAA is to be pulled first, it may have shorter arms connected to this portion, or arms with less slack between them and the hub 170.

In another embodiment, there are 2-4 arms 150 of 35 mm length, each grabbing the distal end of the LAA. There are then 2-4 arms 150′ of 20-30 mm in length, grabbing the middle of the LAA, and 2-4 arms of 5-20 mm in length, each grabbing the proximal portion of the LAA. Thus, the LAA is attached by arms at each portion of its length. In one version of this embodiment there may be a single distal member 130, or there may be multiple distal members 130, 130′, 130″, and 130″′.

In the embodiment of FIG. 2c, a similar result is created by attaching the arms 160 to the distal member 130 at different points along its length.

The arms 150 are pushed into, or expanded into the LAA. The barbs 160 penetrate through the LAA to grab onto the tissue of the LAA. Different mechanisms are utilized. For example, the barb 160 may use a fish hook approach, may utilize a bend 165 to fold the barb backwards to grab the tissue, or another mechanism.

The arms 150 may be cut from a hypotube, be a nitinol wire, or be a bioabsorbable material as described herein. The arms may be designed to push laterally away from the distal member 130, for example a nitinol wire with a preformed bend that takes the distal portion of the arm 150 away from a hub 170 or any other attachment point to the distal member 130.

Typically, the arms are from 10 mm to 1 mm in diameter, or in another embodiment from 2-7 mm in diameter. In a preferred embodiment the arms are from 3-5 mm in diameter.

In use, when the distal member 130 exits the shaft 120, the arms 150 push distally or outwardly from the center, toward the walls of the LAA. Thus, if the distal region 125 of shaft 120 is outside the LAA, the distal member 130 or the arms 150 must advance into the LAA as the arms 150 extend. The location of any preformed bend is thus important, the more distal the preformed bend is, the more quickly the arms 150 will extend laterally toward the wall of the LAA. A preformed bend that is closer to the attachment point or to the hub 170, will result in further advancement before the bent portion of the arm exits the distal region 125 and arm 150 will bend toward the LAA wall.

As shown in FIG. 2c, multiple attachment points for the arms 150 to the distal member 130 are also contemplated, and will allow the arms 150 to exit the distal region 125 of the shaft 120 at different moments, and thus reach to different depths despite potentially having similar lengths or similar bends. Thus, an arm 150 attached to the distal member 130 distally of arm 150′ (not shown) would exit first, and would extend laterally differently than arm 150′.

Once the arms 150 have all extended outward, and barbs 160 have penetrated the LAA wall, the distal member 130 or the arms 150 are pulled back into the left atrium. The LAA is thus inverted and pulled into the left atrium with the device.

The degree to which the LAA is inverted is subject to the goals of the physician. The LAA may be entirely inverted, for example. However, the LAA may be partially inverted. If, for example the LAA is 80% inverted, the LAA tissue may fill the remaining 20% of the LAA, and while still effectively invaginating the LAA, it may not take as much space in the left atrium. Thus, the LAA may be 100% inverted, 90% inverted, 80% inverted, 60% inverted, 40% inverted, 20% inverted, or any amount in between.

Arms 150 may be on separate distal members 130 (not shown). Thus, the distal most extending arms 150 may be on distal member 130, and separately extending arms 150′ may be on distal member 130′, and arms 150″ may be on distal member 130″. This allows a first set of arms to be initially retracted to invert a first portion of the LAA. Then a second set of arms is then retracted to invert a second portion of the LAA, and then the third.

Likewise, the first set of arms 150 and barbs 160 may hook the LAA tissue, and invert a portion of the LAA. Then the second set of arms 150′ is then inserted into the tissue, grabbing it. The first arms 150 may remain in place or be removed. The second set of arms 150′ are moved proximally inverting the second portion of the LAA, and then the third set of arms 150″ and corresponding barbs 160″ are inserted into the tissue, and used to invert the remaining portion of the LAA to be inverted.

If the first set of arms 150 is removed while the second set holds the tissue, they may be reinserted to regrasp the tissue, and further invert the LAA.

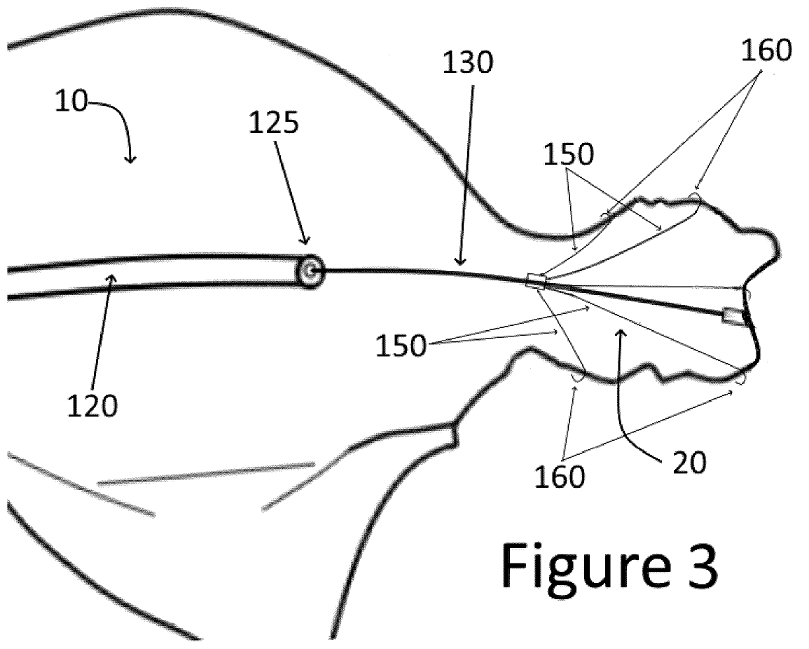

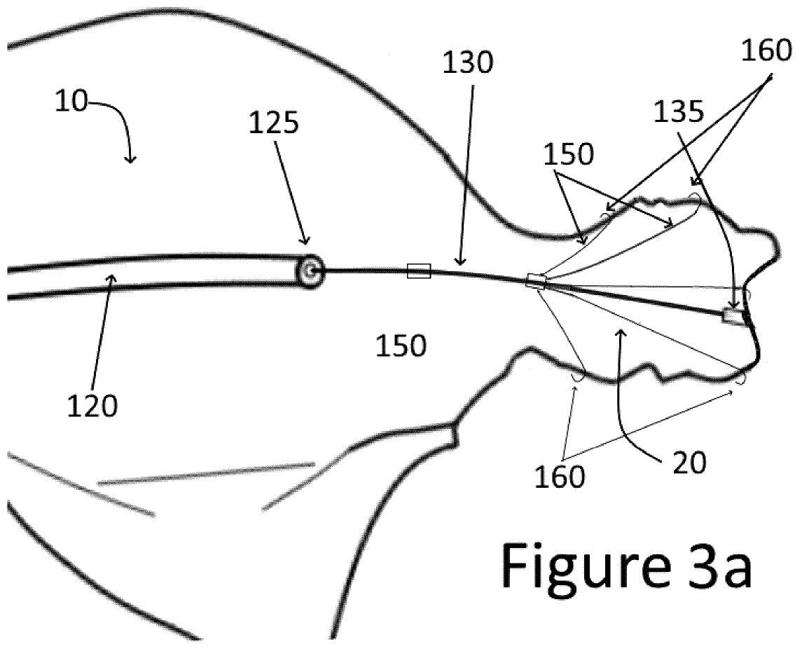

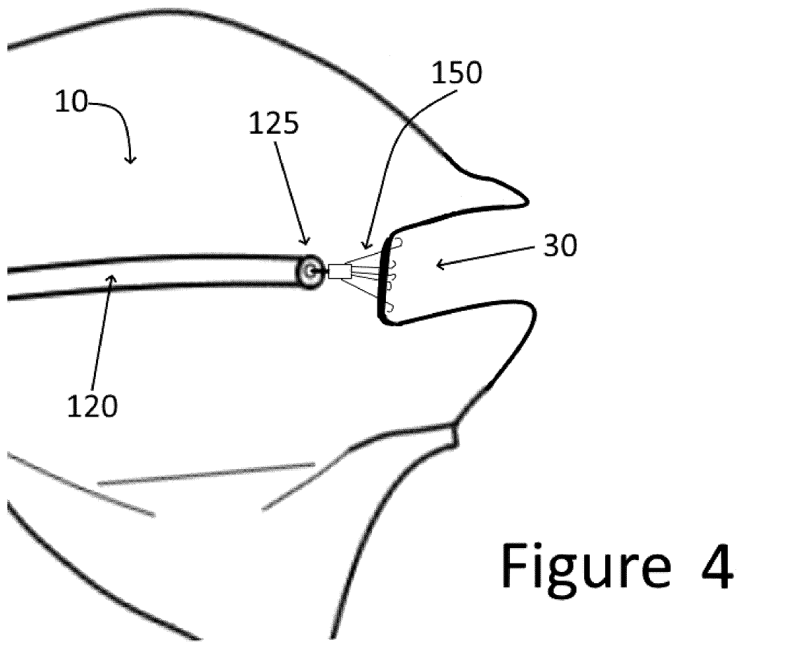

As shown in the progression from FIG. 3, FIG. 3a, and FIG. 4, as the system is withdrawn into the left atrium, the LAA is invaginated to become an inverted or partially inverted LAA 30. Withdrawing the system can comprise withdrawing the entire system together, only withdrawing distal member 130, or activating the arms to withdraw them along distal member 130. Hub 170 may be a collar that slides along distal member 130, and is activated at the handle, e.g., via a pull wire, a spring, a stylus, etc. to slide forward to engage the LAA tissue, and backward to invaginate the LAA.

As shown in FIG. 4, once the LAA is inverted into the left atrium, a looping device is extended from or alongside shaft 120. In one embodiment looping device 200 is a second device introduced in a similar fashion to device 100, via a femoral vein access point for example, and through the fossa ovalis and into the left atrium. For example, sheath 50 may introduce both the shaft 120 and the looping device 200.

In another embodiment the looping device 200 is advanced through the same lumen in shaft 120 that distal member 130 is in. In yet a third embodiment, looping device 200 is within shaft 120's lumen, and distal member 130 advances inside a lumen in looping device 200. Thus, in this embodiment all three shafts are coaxial. In a fourth embodiment looping device 200 and distal member 130 are both in shaft 120, but in separate lumens.

Once the LAA is inverted, looping device is activated. A large suture loop 210 is extended distally. This loop extends around the inverted LAA. The LAA is now inverted, so that the originally distal most tip 220 is now the furthest portion into the left atrium. The loop 210 is extend around and over this originally distal most tip 220 of the LAA. The loop is then advanced distally until it is at or near the ostium of the LAA and the left atrium.

The loop may be constructed of various materials, including non biodegradable materials such as silk, nylon, PTFE, Pet, polyvinlidene fluoride, PEEK, or the like. The loop may also be constructed of resorbable material such as PLLA, PGA, PDLLA, PCL, PLGA, PDLGA, PLDLA, or PLC.

The loop itself is preferably of a large enough loop diameter to extend readily over the inverted LAA and the arms 150 holding it.

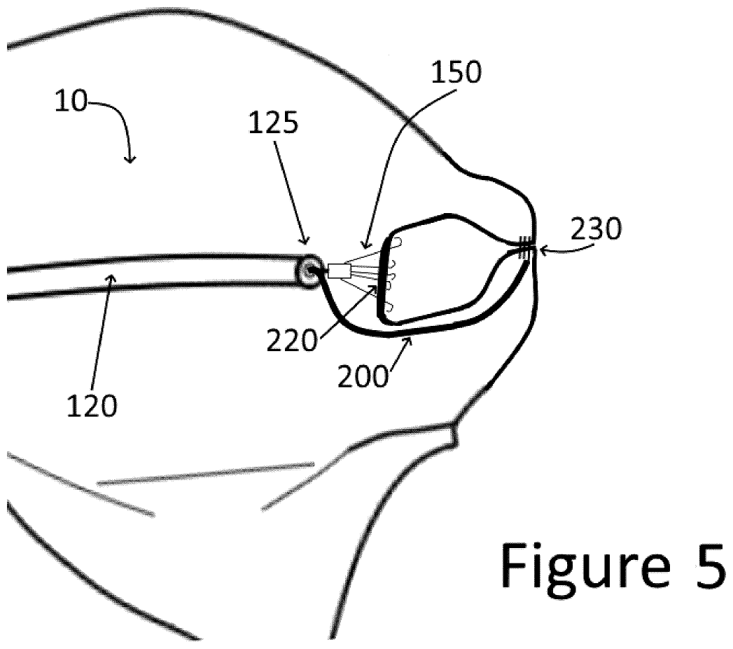

At this point the suture loop 210 is tightened around the LAA, closing it off. As shown in FIG. 5, the suture loop tightens around the LAA and is tied off by looping device 200 to create a tight suture 230 or tight sutures 230. In one embodiment the loop 210 is formed as a snare. At this point suture loop 210 is detached from looping device 200, or is cut, and suture device 200 is removed. If loop 210 is to be cut, looping device 200 may comprise a blade or cutting device on its distal portion for cutting the excess suture material. As such, the suture material may be attached to an actuator for tightening, and may run through or over a cutting blade.

In an alterative embodiment, a suture is wound around the LAA by looping device 200, rather then being placed around it by a preformed loop. In this embodiment, for example, looping device 200 may already be coaxially around the distal member 130 (or distal members 130, 130′, 130″, etc.). By rotating the looping device 200, the suture 210 may spool out of an exit port in the wall of looping device 200 and wind around the LAA into a coil 230 or multiple sutures 230.

Once the suture 210 is in place, it must be fixed in place. For example, the suture may be tied off, a knot, e.g., a preformed knot, may be tightened, or the suture may be sewn into the LAA as needed.

At this point a cutter, e.g., a suture cutter (not shown) is advanced or utilized to cut the remaining suture to size to remove excess material. The cutter may include a slot that the suture is already inside, or a clippers that are moved to engage the excess suture material. To enable this process the suture may include a radiopaque material or marker to make it easily located on x-ray.

In one embodiment the arms 150 and barbs 160 are left in place in the LAA to hold the LAA in place. The arms 150 and barbs 160 may be secured to the suture or a collar to hold the LAA inverted. In this embodiment the arms 150 and barbs 160 may be resorbable into the body, may be comprised of stainless steel, or may be comprised of nitinol, or any combination thereof.

In another embodiment, arms 150 and barbs 160 are detached from the LAA, and removed as well. In some embodiments the barbs 160 can be detached from, unscrewed from, or broken off the arms 150 and are left in place. As such, barbs 160 may be biodegradable or resorbable.

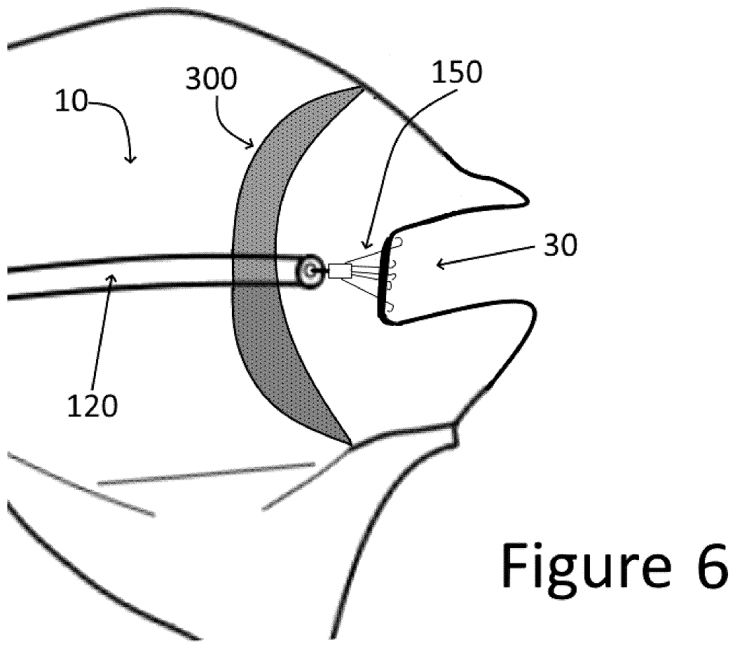

As shown in FIG. 6, in one embodiment a thrombus catching device 300, such as an umbrella, cloth barrier, web, or nitinol structure can be deployed to prevent the release of any thrombus during the procedure. The device 300 would cover the ostium of the LAA, and be large enough that as the LAA is invaginated, it would contain the entire invaginated LAA. Suction may be deployed to remove any thrombus before the system is removed from the patient.

Contrast may be injected to look for any perforations or leaks in the LAA. Imaging technologies such as transesophageal echocardiogram (TEE) and/or Intracardiac echocardiography (ICE) can be used to determine if the LAA is fully invaginated.

In one embodiment the LAA, once sutured and closed off so that it will remain in the left atrium, is then rolled or folded up, and sutured into a smaller form factor.

The anatomy of the LAA can be very irregular. That is, the opening is not formed in a perfect circle. The prior art devices typically use a nitinol structure that is heat set to a specific “round” geometry. When deployed, the nitinol structure can only take the round shape that was defined by the heat setting step. When a prior art device is placed in the LAA, and released, it will only pop open to a predetermined shape, and it will seek to return to that shape if unconstrained. If the ostium of the LAA is not round, which almost is always the case, there is a chance that there will be some portion of the ostium that is not closed off.

Thus, in one embodiment the present invention solves this issue by using grabbers to grab and invert the LAA, a suture to tie off the LAA with a suture, and a catheter system to deliver the device. The device works regardless of the shape of the LAA. In another embodiment, the present invention uses grabbers to grab and partially or fully invert the LAA, and a disk to cover the LAA opening. This embodiment also works regardless of the shape of the LAA. In one subset of this embodiment, in time the disk will be covered by endothelization, and the LAA will be permanently sealed shut. An endothelial layer typically offers an anticoagulant surface, and thus building a layer of endothelial cells over an LAA closure device is advantageous to preventing clot formation.

The different embodiments discussed above and below are related, and use similar elements. So, for example, the LAA grabbers and inverters discussed above, can be used instead of the LAA grabbers discussed below, and vice versa. The handles or catheters discussed in one portion can be used in another embodiment. So the handle discussed above or the coaxial catheter discussed above, for example, could be used with the embodiments below, or the handle discussed below could be used above.

The embodiments discussed herein will preferably include a handle at the proximal end of the system. This handle will include an actuator to unsheathe the device, e.g., by pulling a portion of the handle back to pull a sheath back exposing the distal end of an inner portion, such as an inner catheter, or an actuator that will advance a portion (e.g., an inner catheter) by advancing a portion of the handle, rotating a portion of the handle, or a slider. Another actuator will advance the disk, as described below. The precise number of actuation mechanisms can vary, but one may be present to advance the grabbers, one to extend the grabbers out, one to engage the grabbers, one to pull the grabbers toward the disk, one to invert the LAA with the grabbers, one to advance the disk, one to expand the disk, one to seat the disk, and actuators to release the portion or portions that remain the atrium. One actuator may perform multiple functions, so a single actuator may advance the grabber out, then when further actuated may advance the disk. Upon retraction, that same actuator may pull the grabbers (and thus the LAA) proximally and invert the LAA.

The handle is connected to a sheath 50. The sheath 50 is preferably a double curve sheath, but may also have a single curve and/or have steering wires to guide it to the LAA. In one embodiment the sheath 50 will follow a guidewire from a femoral access point into the heart, through the atrial septum, and to the left atrial appendage (“LAA”). The pre-curves or steering will primarily serve to deliver the device to the LAA.

Preloaded within the sheath is the catheter 120. As the sheath 50 provides the location for the system and presents its distal end to the proper location at the LAA, the catheter 120 does not need to provide steering. In another embodiment, the sheath is not precurved or steerable, and a catheter provides the location by steerability or by being precurved. Once the system is at the desired location the catheter 120 is advanced out of the sheath. In one embodiment its distal end includes a grabber subsystem and a disk subsystem. In another embodiment the catheter 120 may include the disk subsystem, while a second inner catheter provides the grabber subsystem.

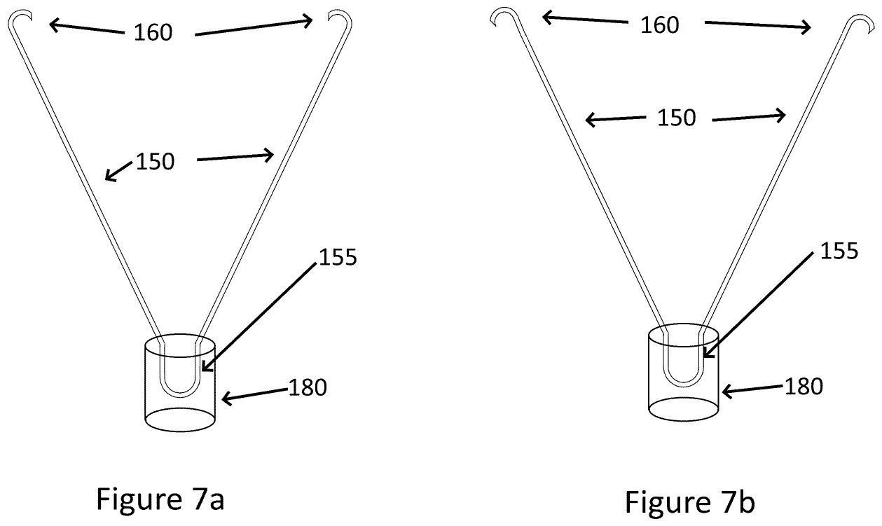

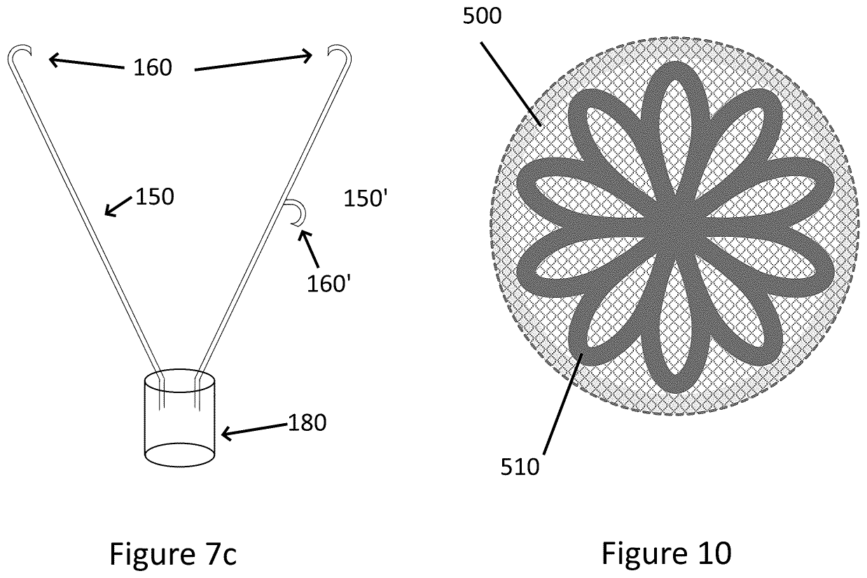

The grabber subsystem will typically include grabbers and fasteners, e.g., hooks, screws, adhesives, or other means described herein for grabbing the LAA. As shown in FIG. 3 the grabbers include arms 150 and barbs/hooks/screws/fasteners 160. As shown in FIG. 7c, the grabbers may each comprise a single arm 150 with a single hook 160, or may comprise a single arm 150′ with multiple hooks 160, 160′. The arms 150 may be attached to a tube 180 (FIG. 7c) or a hub 170 (FIG. 3), or may be freely attached to the inner catheter 130. The attachments can be made via welding, adhesives, friction, or the like. As shown in FIGS. 7a-b, a pair of arms 150 may meet at a bend 155, such as U-shaped bend 155 and thus may be essentially one integral strand with two arms 150 and two hooks 160. If so, the bend may be placed within and secured to tube 150 via a plug, a screw/bolt that screws into the tube 180, or by a hook/loop at the distal end of the catheter 130, tip 135, hub 170. In one embodiment there are grooves in the tube 180 that the grabber arms fit into, and they are secured therein by a plug or a rivet.

In one embodiment the grabber subsystem consists of three pairs of grabbers with two hooks each, arranged each pair as in FIG. 7a or 7b, or some combination thereof (e.g., one pair as in 7a, two pairs as depicted in 7b). The grabbers in FIG. 7a are depicted with the hooks arranged inward, while the grabbers in FIG. 7b are depicted with the hooks arranged outwards. The hooks could also be arranged to one side, upwards, or downwards. Other numerical combinations of grabbers are contemplated, such as three total grabbers, eight grabbers, ten grabbers. While the grabbers are depicted in pairs in FIGS. 7a-c, they could be in groups of three joined at the proximal end, groups of four, etc.

The grabber subsystem is advanced out of the sheath when in the proper location in or near the LAA. The hooks on the distal end are advanced into the LAA tissue, grabbing the tissue and then used to pull it to invert it.

The grabbers may be constructed of numerous medical grade materials, including stainless steel, nitinol, or a resorbable material such as a bioabsorbable polymer such as PLLA, PGA, PDLLA, PCL, PLGA, PDLGA, PLDLA, or PLC. For example, the grabbers arms and hooks may be made of Poly(lactic-co-glycolic acid) PLGA is a biodegradable copolymer ester of two α-hydroxyacides (lactic and glycolic acids and has been approved by the FDA. PLGA nanocarriers undergo degradation by bulk erosion mechanism. If so, the grabber subsystem is left in place at the conclusion of the procedure and is expected to resorb into the body in 12-18 months. In one embodiment, no suction is used to grab onto and manipulate the LAA. The LAA is pulled to or toward the LAA ostium.

Thus, in one embodiment the grabber is constructed from a 1-5″ long, preferably 2-3″ long wire of material such that it comprises two 0.5″-1.5″ arms, preferably 0.75″-1″ long arms. Each arm has a hook formed on the end, and the two arms are joined in the middle by a U-Shaped bend region. The arms may be 0.002-0.035″ thick, preferably 0.004-0.020″ thick, e.g., 0.005″. In particular these dimensions of a resorbable material will allow the grabber arm and hook to be bio absorbed into the body within 6-18 months.

In some embodiments the grabbers are spaced more or less equally around the circumference of the tube 180. However, as the LAA anatomy is irregular, the grabbers may be advantageously adjusted to fit the anatomy. Thus, while regularly spaced grabbers may be best for a more cylindrical or circular LAA, an oblong LAA may be better engaged by grabbers arranged in banks of three on each side, for example. As discussed above, the grabbers may be of different lengths. Likewise, the arm lengths may be adjustable to adapt the length based on the results of an MRI, CT scan, cardiac mapping system (Ensite®, Carto®), or fluoroscopy. For example, as mentioned above the u-shaped portion of the arm pairs depicted in FIGS. 7a and 7b may be attached by one or more hooks/loops at the distal end of the catheter 130. In one embodiment, if each grabber pair is attached by a separate hook, an actuator, connected to a pull wire, may pull the grabber proximally shortening a particular pair, or shortening all of the grabber arms 150.

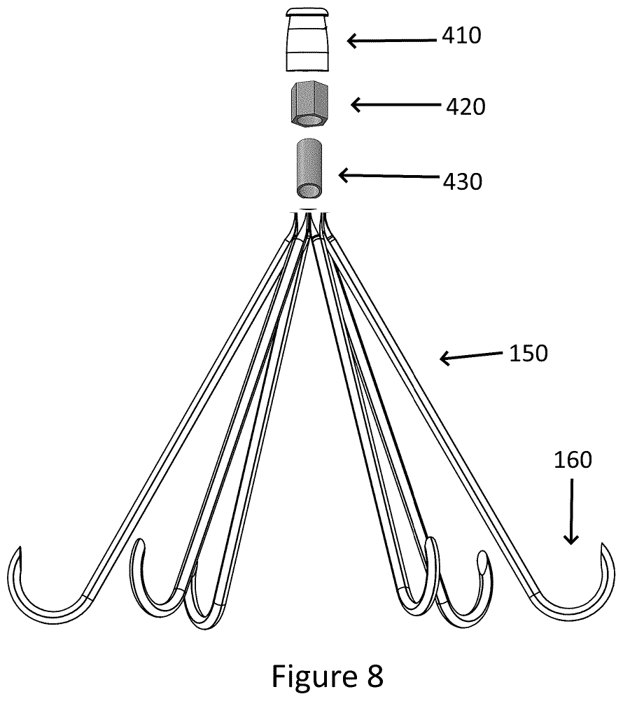

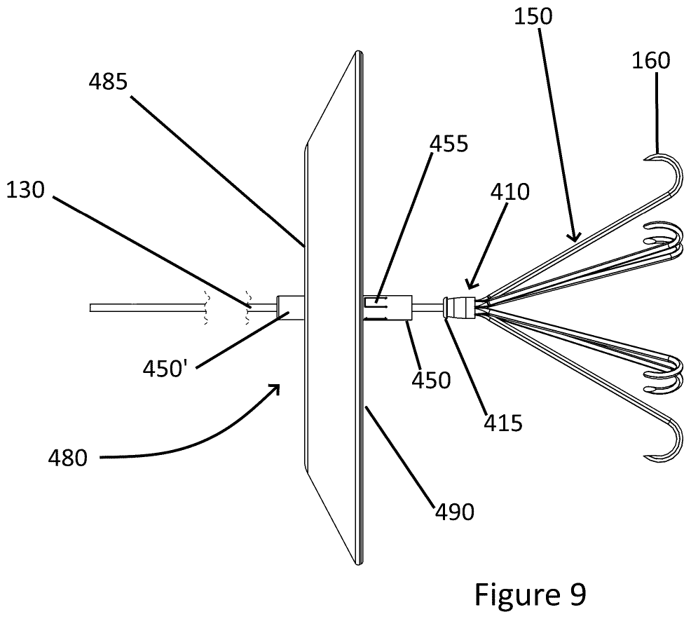

As shown in FIGS. 7a-c, grabber arms 150 may be placed into tube 180. In practice, as shown in FIG. 8, tube 180 may comprise several parts, including a grasper lock 410, a grasper hex mount 420, and a tube 430. For example, the arms 150 may be attached to the tube 430, and hex mount 420 may be slid into the center of tube 430 to secure the arms 150. This assembly is then slid into grasper lock 410. As shown in FIG. 8, the grasper lock 410 is inserted into tube 450. Tube 450 includes catch flaps or notches 455. The lip 415 of grasper lock 410 attaches to the catch flap 455 of tube 450. As shown in FIG. 9, tube 450 is in turn attached to the disk subsystem.

Catheter 130 is attached to one or more of grasper lock 410, hex mount 420, or tube 430. For example, catheter 130 may have a screw on the end, and may be screwed into grasper lock 410. To remove catheter 130 and leave the grabbers in place, the catheter would be rotated to unscrew it (preferably with the grabbers engaged in the tissue, to allow the relative rotation. Catheter 130 is preferably thin, and thus may be a stainless steel hypo tube, nitinol wire, a braided wire, or a suture. Catheter 130 may also be attached to the grasper assembly by a weld, friction fit, plug, or other attachment. The attachment may preferably be broken at a lower force or twist than that required to remove the hooks from the LAA, but higher than required to invert the LAA.

As shown in FIG. 9, the disk subassembly can be attached to catheter 130, shaft 120, or a second catheter 130′. The disk 480 includes a proximal side 485 and a distal side 490. Distal side 490 is preferably attached to tube 450, but tube 450 may extend from distally to proximally of the disk assembly. Alternatively multiple tubes 450, 450′ may attach to the disk assembly.

As shown in FIG. 9, the proximal side 485 may be spaced from the distal side 490, such that there is a cavity within the disk subassembly. Alternatively, the disk may be flat. As shown in FIG. 10, The disk consists of two parts. First, is a fabric layer 500. Second is a disk frame 510. The frame may be the outermost layer, that is most proximal, or the fabric layer may the outermost. The frame may be a braided portion as the frame, and the fabric may be sewn to the frame, welded to the frame, or otherwise adhered.

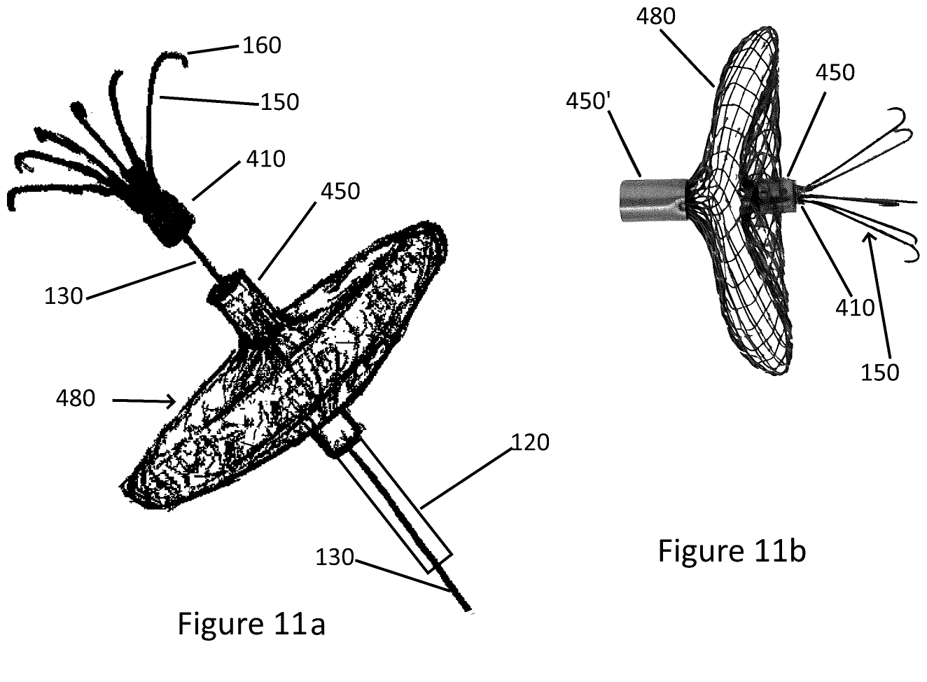

Once the grabber subsystem is in the LAA, the disk subsystem is actuated, expanding and moving the disk from inside the catheter to outside the catheter and into the ostium of the LAA. In a preferred embodiment the disk does not include hooks or points that will penetrate the LAA, the LAA ostium, or other tissue to hold the disk in place. Rather, as shown in FIG. 11a and 11b, the disk is connected to the grabber subsystem. As initially deployed in FIG. 11a, the grabber system 150, 410 is distal of and not connected to the disk subsystem 480. Once the grabber arms and barbs grab the LAA and invert or partially invert it, the hub 410 is pulled back proximally, where the hub 410 engages tube 450, e.g., the hub 410 is inserted into tube 450. In some embodiments, described above, the engagement locks the two together. (FIG. 11b). The LAA pulls the grabber subsystem distally, while the ostium of the LAA pushes the disk subsystem proximally. As the grabber subsystem holds tight to the LAA, the connection between the two holds the disk subsystem in place tightly against the LAA, in the LAA ostium. Thus, as the device is deployed, the grabber subsystem is engaged into the interior tissue of the LAA, This tissue is pulled proximally toward the LA and the LAA ostium. As the disk subsystem is deployed, it covers the ostium. When the two are tightly connected, the LAA tissue pulls the grabber subsystem distally, while the disk subsystem resists that pull by being pushed against the outside of the ostium. As a result, the LAA is retained in or around the ostium, or inside the disk subsystem cavity. As time passes the LA with endothelialize the disk subsystem.

In some embodiments the disk subsystem may also be constructed of bioabsorbable material. In such usage, the material must be resorbed after sufficient endothelization has occurred.

1. A system for deploying a left atrial appendage exclusion device, comprising:
a delivery catheter, the delivery catheter comprising:
a lumen;
an invagination catheter, the invagination catheter comprising:
arms, the arms having barbs for piercing the left atrial appendage;
the invagination catheter inside the lumen of the delivery catheter, and movable relative to the delivery catheter;
a disk assembly comprising:
a fabric material;
a frame, attached to the fabric material;
a disk catheter, the disk catheter removably attached to the frame.

2. The system of claim 1, further comprising a hub, the hub configured to connect the arms to the delivery catheter.

3. The system of claim 2, further comprising a connector, the connector configured to attach the disk assembly to the hub.

4. The system of claim 2, wherein the connector is a tube, the tube having a lip that frictionally mates with a cutout on the disk assembly.

5. The system of claim 4, further comprising a threaded portion on the disk assembly and a threaded portion on the disk catheter, the threaded portion on the disk catheter removably connected to the threaded portion on the disk assembly.

5. The system of claim 1, wherein the barbs are comprised of a bioabsorbable material.

6. The system of claim 5, wherein the arms are comprised of a bioabsorbable material.

7. The system of claim 1, wherein the fabric material is comprised of a bioabsorbable material.

8. The system of claim 7, wherein the frame is comprised of a bioabsorbable material.

9. The system of claim 5, where the bioabsorbable material is a PLLA material.

10. The system of claim 5, where the bioabsorbable material is a PLGA material.

11. The system of claim 1, wherein the invagination catheter is a suture.

12. The system of claim 11, wherein the suture is braided.

13. The system of claim 11, wherein the suture is comprised of a bioabsorbable material.

14. The system of claim 8, wherein the frame is between 20 and 25 mm in diameter.

15. A method of invaginating a left atrial appendage, the method comprising providing
a delivery catheter, the delivery catheter comprising:
a lumen;
an invagination catheter, the invagination catheter comprising:
arms, the arms having barbs for piercing the left atrial appendage;
the invagination catheter inside the lumen of the delivery catheter, and movable relative to the delivery catheter;
a disk assembly comprising:
a fabric material;
a frame, attached to the fabric material;
a disk catheter, the disk catheter removably attached to the frame;
inserting the invagination catheter into the LAA;
piercing the LAA with the barbs
invaginating the LAA by partially removing the invagination catheter proximally covering the ostium of the LAA with the disk assembly.
